(19) United States
(12) Patent Application Publication
(10) Pub. No.: US 2025/0367034 A1
(43) Pub. Date: Dec. 4, 2025

(54) Animal Sound Control Device

(71) Applicant: Hearing Components, Inc., Oakdale, MN (US)

(72) Inventors: Justin Peskar, St. Paul, MN (US); Raymond Ott, III, St. Paul, MN (US)

(21) Appl. No.: 19/215,516

(22) Filed: May 22, 2025

Related U.S. Application Data
(60) Provisional application No. 63/652,293, filed on May 28, 2024.
(60) Provisional application No. 63/736,720, filed on Dec. 20, 2024.

(51) Int. Cl. A61F 11/12; H04R 1/10

(52) U.S. Cl. CPC A61F 11/12; H04R 1/105; H04R 1/1075; H04R 1/1083

(57) ABSTRACT

An animal sound control device, including a housing with a body portion configured to be received within a canine ear canal. The body portion includes a major axis extending between a first end and a second end, and a minor axis extending between a first side wall and a second side wall. The major axis includes a first length greater than a second length of the minor axis.

### CROSS-REFERENCE TO RELATED APPLICATIONS

This application claims the benefit of and priority to U.S. Provisional Application No. 63/652,293 titled “ANIMAL SOUND CONTROL DEVICE,” filed May 28, 2024, and to U.S. Provisional Application No. 63/736,720 titled “ANIMAL SOUND CONTROL DEVICE,” filed Dec. 20, 2024, the contents of which are incorporated by reference herein.

### TECHNICAL FIELD

The subject matter disclosed herein relates to a sound control device, and in particular, to in-ear sound control devices securable to animals (e.g., canines).

### BACKGROUND

In general, sound control devices, such as an ear tip, are designed for humans to deliver audio through the ear canal and/or to attenuate loud noises from the tympanic membrane. Human ear canals are substantially cylindrical, relatively short, and exposed, which enables the human sound control devices to easily seal the ear canal and secure to the ear. For example, a human ear tip generally includes a compliant ear tip configured to seal the ear canal, and in some cases, may include a retention structure to secure to one or more features of the human ear (e.g., an in-ear headphone placed within the concha and/or intertragal notch of the ear).

Sound control devices may be designed for animals to deliver audio through the ear canal and/or to occlude loud noises from the eardrum. For example, a sound control device for canines may provide anxiety control (e.g., from thunderstorms or fireworks), protect canine eardrums from loud noises (e.g., in hunting dogs), and/or enable radio communication from the owner to the canine (e.g., in police or military operations). However, animals have complex ear structures with non-cylindrical ear canals, long ear canals, complex cartilage structures, complex ear muscles, etc., which make it difficult to properly position a sound control device within an animal's ear, to seal an animal's ear canal, and to retain the sound control device within the animal's ear.

### SUMMARY

According to one aspect, an animal sound control device, including a housing with a body portion configured to be received within a canine ear canal. The body portion includes a major axis extending between a first end and a second end, and a minor axis extending between a first side wall and a second side wall. The major axis includes a first length greater than a second length of the minor axis.

In some aspects, the techniques described herein relate to an animal sound control device, including: an ear tip formed of a resilient elastomeric material, the ear tip including an elongate body extending between a proximal end and a distal end; and a housing including a body portion configured to be received within an animal ear canal, the body portion including a major axis extending between a first end and a second end, and a minor axis extending between a first side wall and a second side wall.

### BRIEF DESCRIPTION OF THE DRAWINGS

FIG. 1 is an isometric view of an animal sound control device, according to some embodiments.

FIG. 2A is an isometric front view of a canine ear canal casting, according to some embodiments.

FIG. 2B is an isometric top view of a canine ear canal casting, according to some embodiments.

FIG. 2C is an isometric bottom view of a canine ear canal casting, according to some embodiments.

FIG. 2D is a front diagrammatic view of a canine ear, according to some embodiments.

FIG. 2E is a diagrammatic view of a canine ear canal, according to some embodiments.

FIG. 3A is an isometric view of an ear tip for an animal sound control device, according to some embodiments.

FIG. 3B is an isometric side view of an ear tip for an animal sound control device, according to some embodiments.

FIG. 3C is an isometric side view of an ear tip for an animal sound control device, according to some embodiments.

FIG. 3D is a side view of an ear tip for an animal sound control device, according to some embodiments.

FIG. 3E is a top view of an ear tip for an animal sound control device, according to some embodiments.

FIG. 3F is a side view of an ear tip for an animal sound control device, according to some embodiments.

FIG. 4A is an isometric bottom view of a retention hook secured to an electronics housing for an animal sound control device, according to some embodiments.

FIG. 4B is a cross sectional side view of a retention hook secured to an electronics housing for an animal sound control device, according to some embodiments.

FIG. 4C is an isometric view of a retention hook for an animal sound control device, according to some embodiments.

FIG. 5A is a side view of a first stiffening member for an ear tip of an animal sound control device, according to some embodiments.

FIG. 5B is a side view of a second stiffening member for an ear tip of an animal sound control device, according to some embodiments.

FIG. 6 is a side view of a stiffening member for an ear tip secured to an electronics housing for an animal sound control device, according to some embodiments.

FIG. 7A is an isometric view of an animal sound control device, according to some embodiments.

FIG. 7B is a side view of an animal sound control device, according to some embodiments.

FIG. 8 is flow chart of a method for assembling an animal sound control device, according to some embodiments.

FIG. 9A is an isometric top view of an animal sound control device housing, according to some embodiments.

FIG. 9B is a top view of the housing of the animal sound control device with the cover removed, according to some embodiments

FIG. 9C is a front side view of the second end of the housing, according to some embodiments.

FIG. 9D is a diagrammatic top view of a cross-sectional profile of the housing within a canine ear canal at a tragus-antihelix plane, according to some embodiments.

FIG. 10A is an isometric front view of an animal sound control device, according to some embodiments.

FIG. 10B is an isometric rear view of the animal sound control device, according to some embodiments.

FIG. 11A is an isometric front view of an animal sound control device, according to some embodiments.

FIG. 11B is an isometric rear view of the animal sound control device, according to some embodiments.

FIG. 12A is a side view of an ear tip for an animal sound control device, according to some embodiments.

FIG. 12B is a bottom view of the ear tip of an animal sound control device, according to some embodiments.

FIG. 12C is an isometric side view of the ear tip for an animal sound control device including a stiffening member and a sound tube, according to some embodiments.

FIG. 12D is a side view of the stiffening member of the ear tip, according to some embodiments.

### DETAILED DESCRIPTION

The present disclosure describes an animal sound control device for use on an animal (e.g., a canine). The animal sound control device includes an ear tip configured to seal the ear canal of the animal. The ear tip is formed of a compliant material (e.g., a viscoelastic foam) and includes a sealing head which forms a substantially continuous seal around the circumference of the ear canal. The ear canal of the animal may have complex (e.g., non-cylindrical) shapes extending deep into the ear structure. In some embodiments, the sealing head is angled relative to the elongate body of the ear tip to seal a bend (e.g., the region between the vertical canal and the horizonal canal of a canine) in the ear canal. In order to properly seat the sealing head within the ear canal, the animal sound control device includes a stiffening rib to provide structural stiffness to the ear tip. The animal sound control device includes a housing secured to the ear tip and/or the stiffening rib. In some embodiments, the housing includes electronics components, including for example, a microphone, a speaker, and/or an audio connection port. The electronics components may deliver audio to the animal through a sound tube extending through the ear tip and/or the stiffening rib. The housing is secured in-car via a retention hook. The retention hook includes one or more features to engage the inner ear of the animal (e.g., an antihelix hook and/or a tragus shoe).

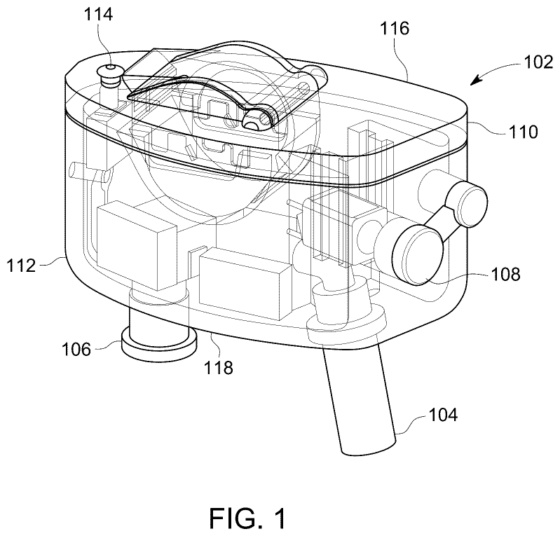

FIG. 1 is an isometric view of an animal sound control device 100 (i.e., an animal hearing component), according to some embodiments. In some embodiments, the animal sound control device 100 includes an electronics housing 102, an audio port 104 and a detent 106 positioned at a second end 118 of the electronics housing 102, audio input port 108, and a cap 110 and a microphone 114 positioned at a first end 116 of the electronics housing 102.

The electronics housing 102 includes a cavity configured to house one or more electronic components therein. For example, the one or more electronic components include a battery, a microphone, a speaker, and/or an electronic control unit (ECU). The audio input port 108 may be optionally configured to receive an audio jack. The one or more electronics components housed within the electronics housing are configured to output audio signals (e.g., from the microphone 114 disposed within the electronics housing 102 and/or from the audio input port 108) through the audio port 104 to an ear canal of an animal (e.g., a canine). For instance, the microphone 114 disposed on or within the electronics housing 102 detects audio and relays the detected audio to the speaker which delivers audio to the audio port 104. In some embodiments, the ECU (not shown) modifies the magnitude of the audio signal (e.g., amplifies the audio or reduces the audio). For instance, the ECU includes an acoustic filter to reduce maximum audio level and/or attenuate impulse noises (e.g., the microphone 114 receives external audio and the ECU filters the audio). In some embodiments, the audio input port 108 is coupled to an external device (e.g., radio, telecommunication, Bluetooth, etc.) to enable remote communication with the animal.

In some embodiments, the body portion 112 of the electronics housing 102 is secured to the cap 110 via a fastener (not shown) removably securable to the electronics housing 102 to allow the cap 110 to be selectively moved for maintenance of the animal sound control device (e.g., replacing the battery). In some embodiments, the cap 110 includes a tab to facilitate grasping for removal of the animal sound control device 100. The electronics housing 102 is formed of a ductile polymer to provide shatter resistance.

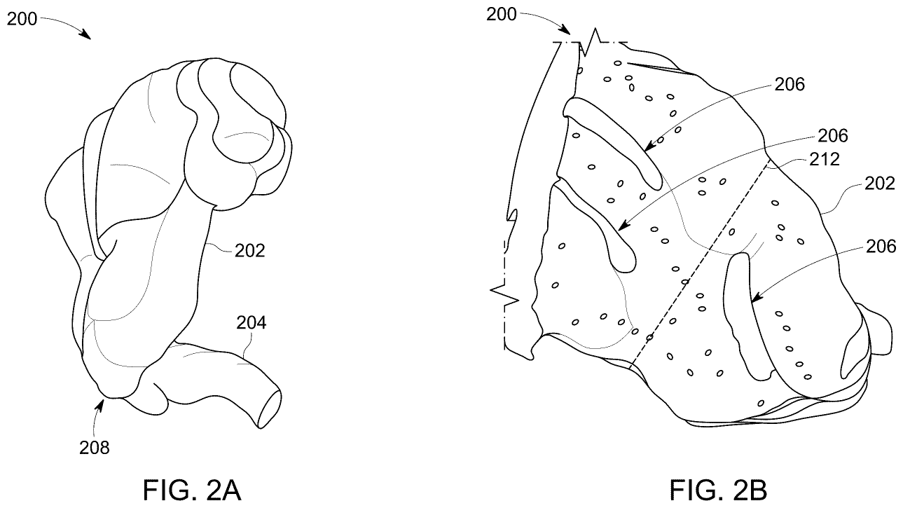

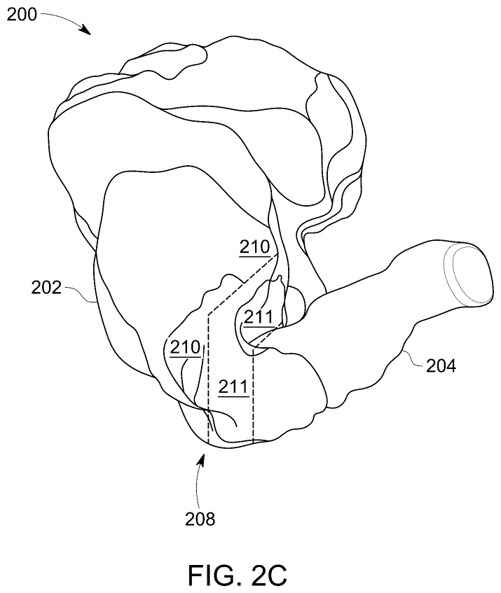

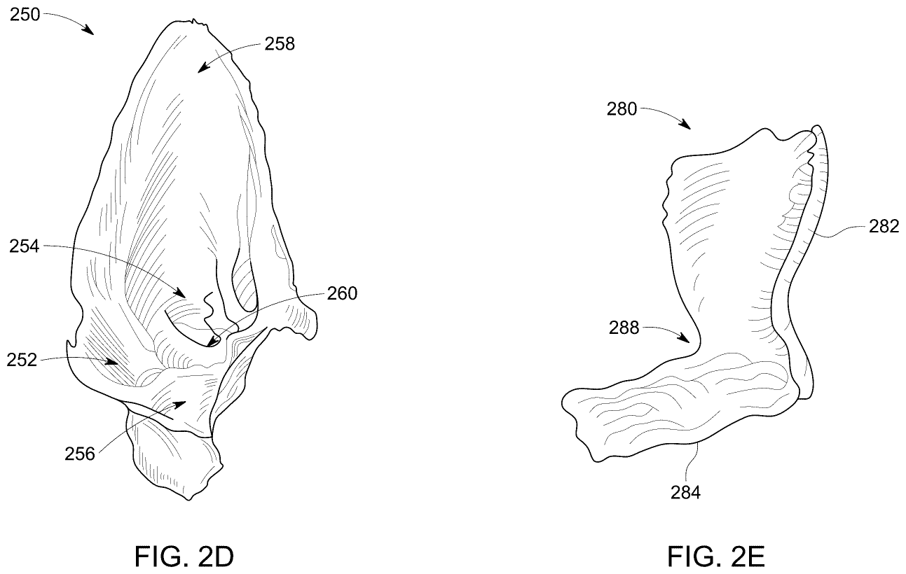

FIGS. 2A-C are isometric views of a canine ear canal 200, according to some embodiments. It should be noted that the animal sound control device(s) described herein are not limited to canine applications, but rather, can be used on various animals having similar ear canal structures (e.g., cats or horses). The canine ear canal 200 includes a vertical canal 202 and a horizontal canal 204. A bend 208 is positioned between the vertical canal 202 and the horizontal canal 204. One or more ridge features 206 are present in the canine ear canal 200.

FIG. 2A is a rear isometric view of the left canine ear canal casting 200, according to some embodiments. In other words, the left canine ear canal casting 200 is a mold of a canine ear canal, i.e., the left canine ear canal casting 200 is a negative of the ear canal. The vertical canal 202 extends in a vertical direction from the pinna (ear flap) down into the inner ear structure. The vertical canal 202 includes a plurality of ridge features 206 (or complex, non-cylindrical structures). The ridge features 206 have high aspect ratios (i.e., the ratio of its sizes in different dimensions), as for instance, the ridge feature 206 is a deep ridge having a short distance between adjacent walls. The ridge features 206 are difficult to seal with a compliant ear tip. For example, an expandable foam tip may be unable to extend to the apex of the ridge, and therefore, the ridge features 206 permit sound to circumvent an ear tip. The bend 208 (also referred to as the 90° bend) is a transition region between the vertical canal 202 and the horizonal canal 204. The horizonal canal 204 extends inward (toward the skull) from the bend 208. The horizonal canal 204 includes complex (high-aspect ratio) structures and rapidly decreases in diameter as it extends toward the skull.

It should be noted that human ear canals lack the complex, non-cylindrical structures of the vertical canal 202. Human ear canals lack the 90° bend between the vertical canal 202 and the horizontal canal 204, and instead, human ear canals merely include a substantially cylindrical and accessible horizonal canal. Thus, the canine car canal 200 is more difficult to properly seal than a human ear canal.

FIG. 2B is a top isometric view of the left canine ear canal casting 200, according to some embodiments. The vertical canal 202 includes the plurality of ridge features 206 preventing a proper seal from a compliant ear tip. However, the vertical canal 202 includes an intermediate region (secondary sealing plane 212) with minimal complex features.

FIG. 2C is a bottom isometric view of the left canine ear canal casting 200, according to some embodiments. The bend 208 includes a region with minimal complex features (i.e., a region with a low aspect ratio features). A first sealing plane 210 is illustrated across the region above the bend 208. An alternative sealing plane 211 is illustrated across the region below the bend 208.

FIG. 2D is a front diagrammatic view of a canine ear 250, according to some embodiments. The canine ear 250 includes an antitragus 252, a concha 254, a tragus 256, a pinna 258, and an antihelix 260, according to some embodiments. The antitragus 252, the concha 254, the tragus 256, the pinna 258, and/or the antihelix 260 are semi-flexible cartilage features of the canine ear 250.

FIG. 2E is a diagrammatic view of a canine ear canal 280, according to some embodiments. The canine ear canal 280 includes a vertical canal 282 and a horizontal canal 284 with a bend 288 separating the vertical canal 282 from the horizontal canal 284, according to some embodiments.

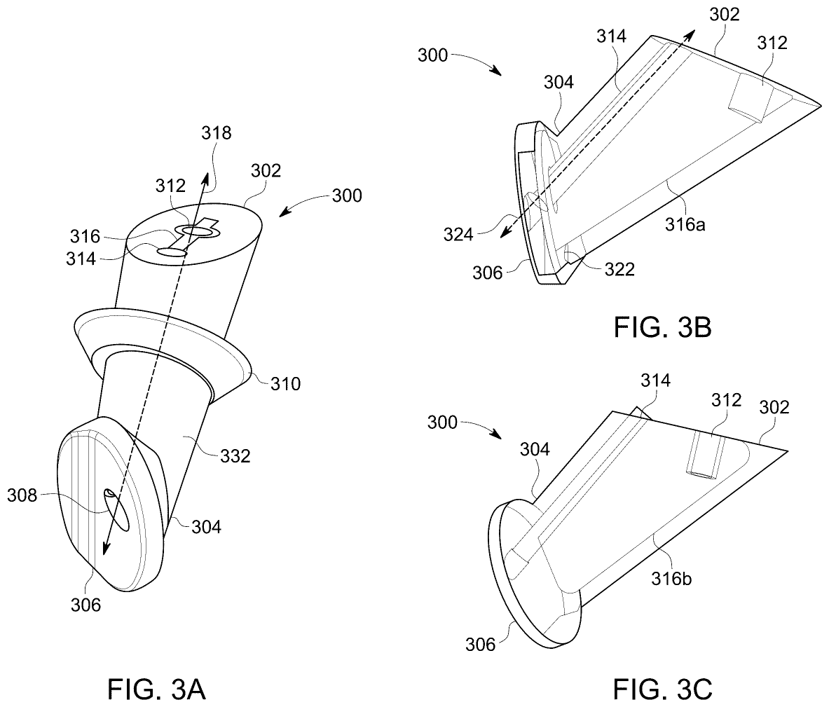

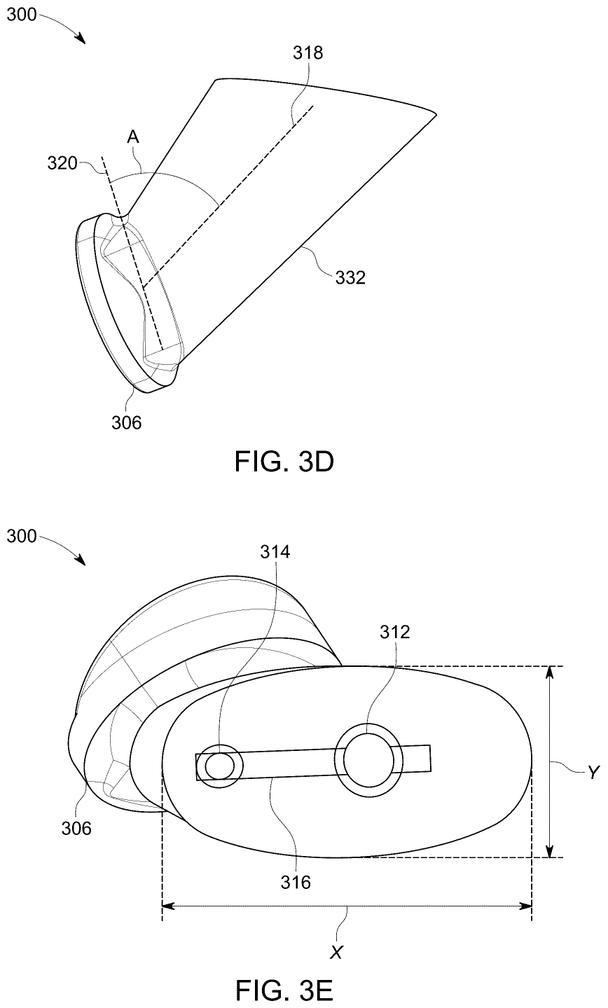

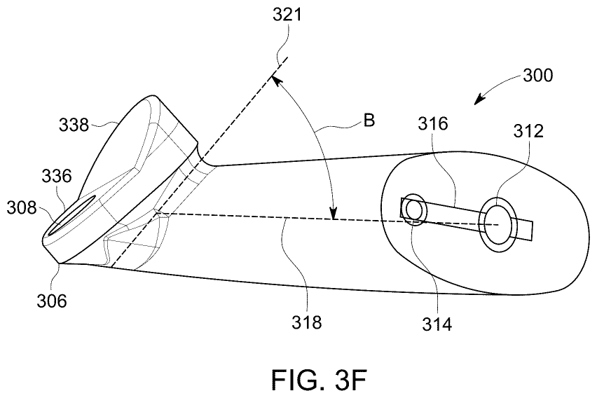

FIG. 3A is an isometric view an ear tip 300 for an animal sound control device, according to some embodiments. In some embodiments, the ear tip 300 includes a proximal end 302 and a distal end 304 with an elongate body 332 extending therebetween along a cylindrical axis 318, a sealing head 306 forming a sealing plane, an audio transmission aperture 308, and a concentric barb 310. In some embodiments, the ear tip 300 includes a stiffening member 316 including an anchor port 312, a sound tube 314 extending along a sound tube axis 324, and a stiffening rib 328. The ear tip 300 is formed of a compliant material, including for example, a resilient foam and/or a viscoelastic foam. The stiffening member 316 is formed of a semi-rigid material configured to provide rigidity along the cylindrical axis 318 and/or the sound tube axis 324 during insertion of the ear tip 300 within the animal ear canal (e.g., within the canine ear canal 200). If, for example, the ear tip 300 was formed solely from the compliant material (e.g., resilient foam), it may be difficult to insert the ear tip 300 to the correct position/depth of the animal ear canal, as the ear tip 300 may compress axially along the cylindrical axis 318. The stiffening member 316 limits axial compression of the ear tip to ensure the sealing head 306 of the ear tip 300 reaches the proper position (e.g., the primary sealing plane 210) within the animal ear canal.

FIG. 3B is a side view of an ear tip 300 of an animal sound control device, according to some embodiments. In some embodiments, the ear tip 300 includes a first stiffening member 316a with a stiffening head 322. FIG. 3C is a side view of an ear tip 300 of an animal sound control device, according to some embodiments. In some embodiments, the ear tip 300 includes a second stiffening member 316b.

In some embodiments, the sealing head 306 of the ear tip 300 is configured to form a complete and continuous seal around the circumference of an animal ear canal. For instance, in some embodiments the sealing head 306 is positioned to form a seal at the primary sealing plane 210 shown in FIG. 2C. The primary sealing plane 210 is located at or near the bend 208, and therefore, the primary scaling plane 210 is angled non-orthogonally relative to the vertical canal 202. The sealing head 306 is likewise angled non-orthogonally relative to the sound tube axis 324 to follow the path of the animal ear canal into the bend 208. The sealing head 306 is positioned on the elongate body 332 of the ear tip 300. In some embodiments, the sealing head 306 is positioned at the distal end 304 of the ear tip 300. In some embodiments, the sealing head 306 is positioned between the proximal end 302 and the distal end 304 of the ear tip.

FIGS. 3D-F are views of the ear tip 300, according to some embodiments. FIG. 3A is a side view of the ear tip 300 having the cylindrical axis 318 (e.g., an axis extending along the elongate body 332) and the sealing head 306. The sealing head defines a scaling head plane, having a longitudinal dimension 320 (see FIG. 3D) and a lateral dimension 321 (see FIG. 3F).

In some embodiments, the longitudinal dimension 320 of the sealing head plane is oriented at an angle A relative to the cylindrical axis 318 of the ear tip 300. The angle A is defined within a range of between 80° and 20°, and in some embodiments, the angle A is defined within a range of between 75° and 40°, and in some embodiments the angle A is between 51° and 71°. The longitudinal dimension 320 of the sealing head plane is important, as it orients the sealing head 306 to seal an animal ear canal near the 90° bend. For instance, longitudinal dimension 320 of the sealing head plane may be configured to match an angle of the primary sealing plane 210 shown in FIG. 2C.

In some embodiments, the lateral dimension 321 of the sealing head plane is oriented at an angle B relative to the cylindrical axis 318 of the ear tip 300. The angle B is defined within a range of between 90° and 30°, and in some embodiments, the angle B is defined within a range of between 80° and 35°, and in some embodiments the angle B is between 67° and 47°. The lateral dimension 321 of the sealing head plane is important, as it orients the scaling head 306 to seal an animal ear canal near the 90° bend. For instance, lateral dimension 321 of the sealing head plane may be configured to match an angle of the primary scaling plane 210 shown in FIG. 2C.

In some embodiments, the sealing head 306 includes a plurality of planar surfaces. For example, the sealing head 306 shown in FIG. 3F includes a first planar surface 336 and a second planar surface 338. The audio transmission aperture 308 extends through the first planar surface 336, according to some embodiments. The plurality of planar surfaces 336, 338 improves the seal of the sealing head 306, as in some cases, one or more of the plurality of planar surfaces 336, 338 extend(s) at least partially into the 90° bend region of an animal ear canal.

In some embodiments, shown in FIG. 3E, the proximal end 302 of the ear tip 300 includes a length dimension X and a width dimension Y. The length dimension X and/or the width dimension Y continuously decreases along the elongate body 332 from the proximal end 302 to the distal end 304 (until the sealing head 306 is reached), according to some embodiments. For example, the length dimension X and/or the width dimension Y are reduced by between 10% and 50% from the proximal end 302 to the distal end 304 (not including the sealing head 306). In some embodiments, the length X to the width Y ratio is between 3:1 and 5:4, and in some embodiments the length X to the width Y ratio is between 5:2 and 3:2. In some embodiments, the width Y is less than a width of the sealing head 306. In other words, the width of the sealing head 306 is greater than the width Y of the elongate body 332.

In an exemplary embodiment of an ear tip designed for canine ear canals (e.g., canines of 40+ lbs., according to some embodiments), the length X of the proximal end 302 is within a range of between 1.50 inches and 0.75 inches, the width Y of the proximal end 302 is within a range of between 0.75 inches and 0.30 inches, the length X of the distal end 304 (not including the sealing head 306) is within a range of between 1.25 inches and 0.40 inches, the width Y of the distal end 304 (not including the sealing head 306) is within a range of between 0.60 inches and 0.20 inches, the length X of the sealing head 306 is within a range of between 1.25 inches and 0.50 inches, and the width Y of the sealing head 306 is within a range of between 1.25 inches and 0.50 inches.

In some embodiments, the sealing head 306 includes a diameter (e.g., across the sealing head plane) that is greater than a diameter of the distal end 304 of the elongate body 332. In other words, the sealing head 306 flares outward from the distal end 304 to cover a greater cross sectional area. In some embodiments, the proximal end 302 of the elongate body 332 has a greater diameter than the distal end 304 of the elongate body 332. The cross sectional diameter of the elongate body 332 continuously decreases from the proximal end 302 (maximum diameter) to the distal end 304 (minimum diameter), according to some embodiments. The diameter of the sealing head 306 is less than the diameter of the proximal end 302, according to some embodiments.

In some embodiments, the concentric barb 310 is positioned on the elongate body 332 between the proximal end 302 and the distal end 304. The concentric barb 310 extends radially outward from the elongate body 332 to contact the vertical canal of an animal ear canal. In some embodiments, the contact between the concentric barb 310 and the vertical canal provides stability to the ear tip 300, i.e., the contact between the concentric barb 310 and the vertical canal limits the movement of the ear tip 300 within the ear canal ensure the sealing head 306 remains in-place. The contact between the concentric barb 310 and the vertical canal provides structural support for the elongate body 332 against the animal ear canal. For example, if an animal has a wide vertical canal, the elongate body 332 may be unsupported between the proximal end 302 and the distal end 304. The concentric barb 310 helps ensure contact between the elongate body 332 and the vertical canal to support the ear tip 300.

In some embodiments, the concentric barb 310 is configured to form a secondary seal of the animal ear canal. For example, the concentric barb 310 can be positioned at the secondary sealing plane 212 as shown in FIG. 2B to form a continuous sealing across the animal ear canal. The primary and secondary seal (the sealing head 306 and the concentric barb 310, respectively) provides a robust seal of the animal ear canal. The concentric barb 310 occludes a portion of the acoustic waves entering the animal ear canal and anchors/stabilizes the elongate body 332 to ensure the sealing head 306 is properly positioned and supported. The sealing head 306 occludes acoustic waves at the bottom portion of the vertical canal and/or at the 90° bend.

In some embodiments, the concentric barb 310 is angled toward the proximal end 302 of the elongate body. For instance, the concentric barb 310 is secured to the elongate body 332 and flares upward and radially outward. The angle of the concentric barb 310 is configured to minimize friction upon insertion of the ear tip 300 within an animal ear canal. The uppermost (proximal) portion of the concentric barb 310 is cantilevered (supported at the distal portion and unsupported at the proximal portion). The cantilevered configuration is beneficial because it provides a resilient, compressible surface to engage the ear canal.

In some embodiments (see e.g., FIG. 3B), the stiffening member 316a includes a stiffening head 322. The stiffening head 322 is disposed within the sealing head 306 and configured to provide structural support for the sealing head 306. The sound tube 314 extends though (i.e., distal to) the stiffening head 322, according to some embodiments.

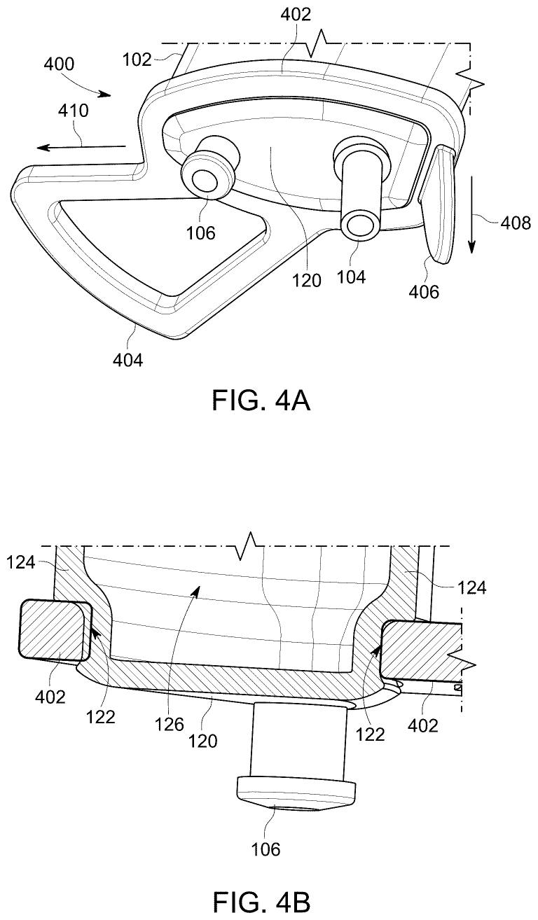

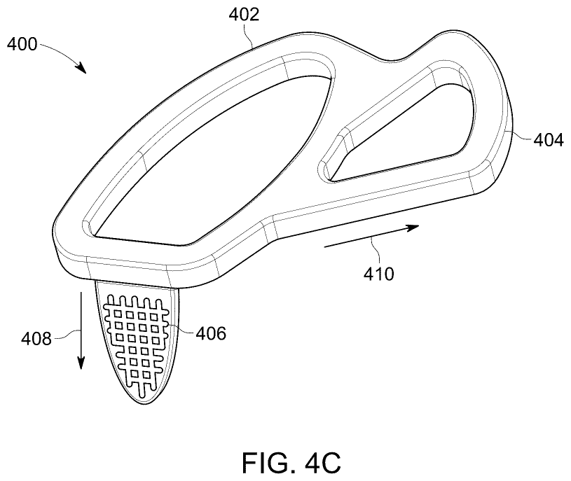

FIG. 4A is an isometric bottom view of a retention hook 400 secured to an electronics housing 102 for an animal sound control device, according to some embodiments. The retention hook 400 includes a retention ring 402, a wing 404 extending in a lateral direction 410, and a canal heel 406 extending in a distal direction 408. FIG. 4B is a cross sectional side view of the retention hook 400 secured to the electronics housing 102 for an animal sound control device, according to some embodiments. The retention ring 402 is received within a channel 122 formed by a housing wall 124. The housing wall 124 of the electronics housing 102 forms a cavity 126 configured to house one or more electronics components. FIG. 4C is an insolated isometric view of the retention hook 400.

In some embodiments, the retention hook 400 stabilizes and secures the electronics housing 102 (and the components secured to the electronics housing 102, e.g., the ear tip 300 and/or the stiffening member 316) in an animal ear. The wing 404 includes an antihelix hook. In other words, the wing 404 is configured to engage an antihelix of an animal ear (e.g., an antihelix of a canine). The wing 404 extends in the lateral direction 410 substantially parallel to the distal face 120 of the electronics housing 102. The canal heel 406 includes a tragus shoe configured to engage a tragus of an animal ear (e.g., a tragus of a canine). The canal heel 406 extends in the distal direction 408 substantially orthogonal to the wing 404. In some embodiments, the canal heel extends at an angle of approximately 80° from the wing 404. The retention ring 402 is positioned between the wing 404 and the canal heel 406.

In some embodiments, the wing 404 and/or the canal heel 406 limits the travel of the animal sound control device 100 in the animal ear. For example, the wing 404 and/or the canal heel 406 engages the cartilage of the animal ear to prevent the animal sound control device 100 from moving lateral within the ear, from rotating within the ear, and/or from pulling out of the ear. The position of the retention ring 402 (and therefore, the electronics housing 102 and the ear tip 300) between the wing 404 and the canal heel 406 provides stability to opposing sides of the retention ring 402, thereby improving stability of the animal sound control device 100 within the animal ear. In other words, the wing 404 and the canal heel 406 provide diametrically opposed anchor points to hold the animal sound control device in-place. The multiple extension planes of the wing 404 and the canal heel 406 (the wing 404 extending in the lateral direction 410 and the canal heel 406 extending in the distal direction 408) improves the stability of the animal sound control device 100 within the animal ear.

In some embodiments, the retention ring 402 is received within the channel 122 of the electronics housing. The retention ring 402 is formed of an elastic and/or resilient material configured to stretch around the distal face 120 of the electronics housing 102 and snap into the channel 122. The retention ring 402 is removably secured to the electronics housing 102. For instance, the retention hook 400 can be removed and replaced. Such configuration is beneficial, as different retention hooks can be swapped out to customize the fit for different types/sizes of animals. For example, the retention hook 400 shown in FIG. 4A is an exemplary embodiment for a canine weighing 80+ pounds. However, a second retention hook (not shown) can be secured to the electronics housing 102 for a canine weighing between 41-50 pounds.

In some embodiments, the retention hook 400 is formed of a compliant material (e.g., an elastic silicon). The compliant material must provide enough structural strength to hold the animal sound control device 100 in-place, but cannot be too rigid as to cause discomfort to the animal. In some embodiments, the retention hook 400 is formed of a compliant material having between 50-80 Shore A hardness. In some embodiments, the retention hook 400 includes an internal frame with the complaint material over molded onto the frame. In some embodiments, the retention hook 400 is formed of the same and/or similar material to the ear tip 300.

In some embodiments, the wing 404 and/or the canal heel 406 include gaps or regions with no material within the respective perimeters of the wing 404 and the canal heel 406 to allow for variability in the structure of the animal antihelix, which occasionally has protrusions or other deformations to the surface. The open space permits the wing 404 and/or the canal heel 406 to conform to irregularities. Furthermore, the open space(s) or gap(s) permits the person inserting the device into the animal ear to bend the wing 404 and/or the canal heel 406 to the proper position without causing the animal discomfort. The gaps/open spaces of the wing 404 and/or the canal heel 406 allow the retention hook 400 bend or deform to the shape of the animal ear canal without sacrificing grip, interference, or strength of the device.

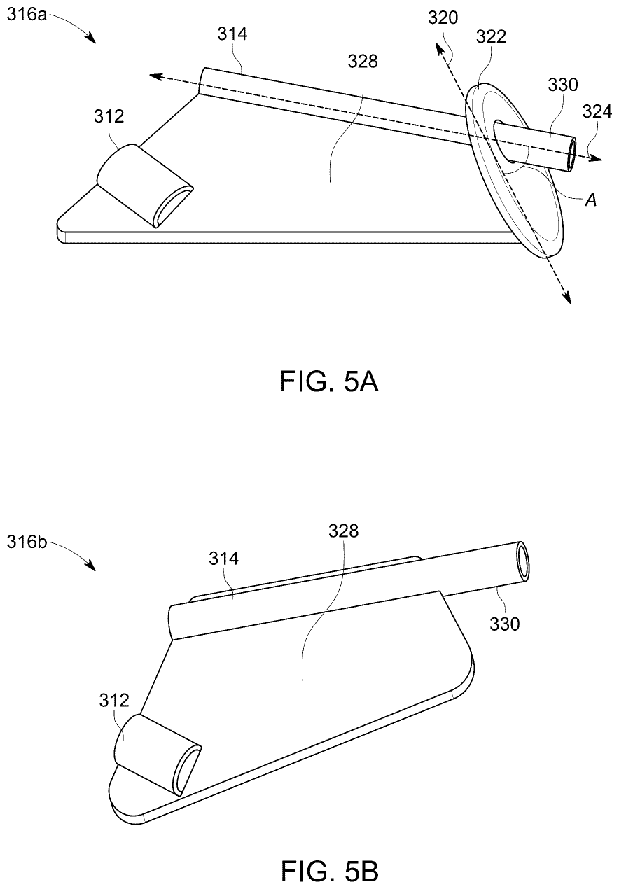

FIG. 5A is a side view of a first stiffening member 316a for an ear tip 300 of an animal sound control device 100, according to some embodiments. The first stiffening member 316a includes the sound tube 314 having the sound tube axis 324, the anchor port 312, the stiffening rib 328, the stiffening head 322 positioned on the sealing head plane, and a cantilevered portion 330 of the sound tube 314 extending beyond the stiffening head 322, according to some embodiments. The stiffening rib 328 extends from the sound tube 314 to the stiffening head 322 and from the anchor port 312 to the stiffening head 322. The stiffening rib 328 is substantially thin, as for example, the thickness of the stiffening rib 328 is less than the diameter of the sound tube 314. The stiffening rib 328 is compliant in a lateral direction but stiff along an axial direction (i.e., in the direction of the sound tube axis 324). The stiffening rib 328 provides structural support for the ear tip 300 during insertion of the ear tip 300 into the animal ear canal. In some embodiments, the cantilevered portion 330 of the sound tube 314 has greater flexibility than the upper portion of the sound tube 314.

FIG. 5B is a side view of a second stiffening member 316b for an ear tip 300 of an animal sound control device 100, according to some embodiments. The second stiffening member 316b includes the sound tube 314, the anchor port 312, the stiffening rib 328, and a cantilevered portion 330 of the sound tube 314 extending beyond the stiffening rib 328, according to some embodiments.

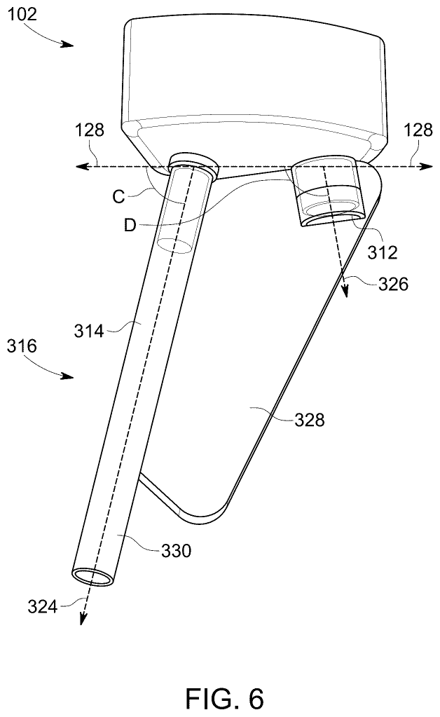

FIG. 6 is a side view of a stiffening member 316 for an ear tip 300 secured to an electronics housing 102 for an animal sound control device 100, according to some embodiments. The stiffening member 316 includes the sound tube 314 extending along the sound tube axis, the anchor port 312 extending along an anchor port axis 326, the stiffening rib 328, and a cantilevered portion 330 of the sound tube 314 extending beyond the stiffening rib 328, according to some embodiments. The electronics housing 102 includes a distal face axis 128. In some embodiments, an angle C is formed between the distal face axis 128 and the sound tube axis 324 and an angle D is formed between the distal face axis 128 and the anchor port axis 326. The angle C is non-orthogonal and/or the angle D is non-orthogonal, according to some embodiments. The angle C is not equal to the angle D. and in some embodiments, the angle C is acute whereas the angle D is obtuse. The different angles of the sound tube 314 and the anchor port 312 form a stronger connection between the electronics housing 102 and the stiffening member 316 and more stable base for the ear tip 300. In some embodiments, the sound tube 314 and the anchor port 312 are different sizes/geometries (corresponding to the sizes of the audio port 104 and the detent 106) to ensure the stiffening member 316 is not secured in reverse.

In some embodiments, the stiffening member 316 is removably securable to the electronics housing 102. For example, the detent 106 includes a ridge, rib, or raised feature to snap onto the anchor port 312. In some embodiments, the audio port 104 includes a ridge, rib, or raised feature to snap onto the sound tube 314. In some embodiments, the distal face 120 includes one or more features to engage the stiffening member 316. The stiffening member 316 is removably securable via a snap on/snap off feature (i.e., a friction fit or force fit between the anchor port 312 and the detent 106 and/or between the sound tube 314 and the audio port 104). Removing the stiffening member 316 is beneficial, as for example, the stiffening member (and ear tip attached thereto) can be replaced or swapped out to customize the fit for different types/sizes of animals.

The two-point connection (i.e., the anchor port 312 and the sound tube 314) is beneficial because it spreads the load forces (i.e., lateral forces upon the ear tip) across multiple connection points. For instance, if the sole connection point was the sound tube 314, the audio port 104 would bear all load forces acting upon the ear tip. The two-point connection (i.e., the anchor port 312 and the sound tube 314) provides a stable connection between the electronics housing 102 and the stiffening member 316.

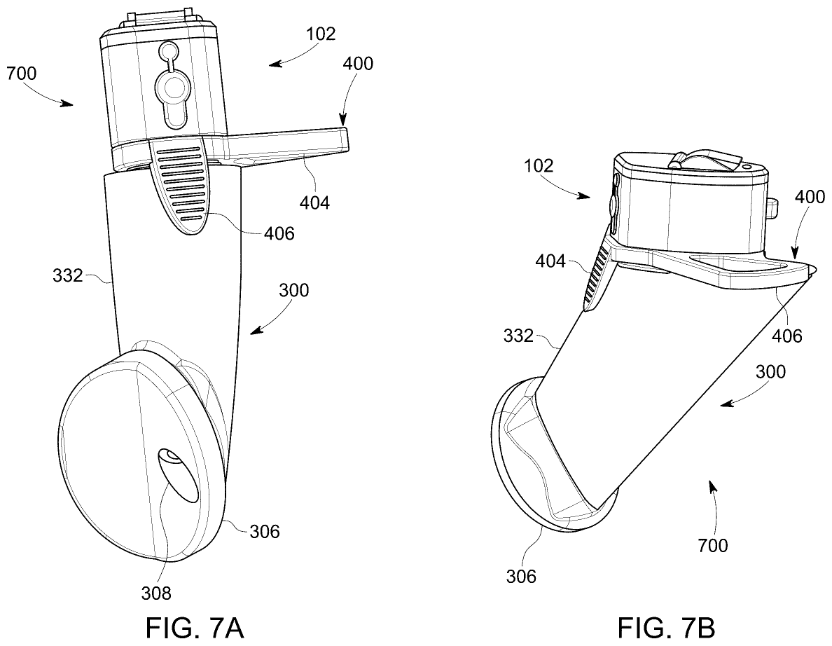

FIGS. 7A-B are views of an animal sound control device 700, according to some embodiments. The animal sound control device 700 includes the electronics housing 102 (with any and/or all features of the electronics housing 102 described above), the retention hook 400 (with any and/or all features of the retention hook 400 described above), and the ear tip 300 (with any and/or all features of the ear tip 300 described above).

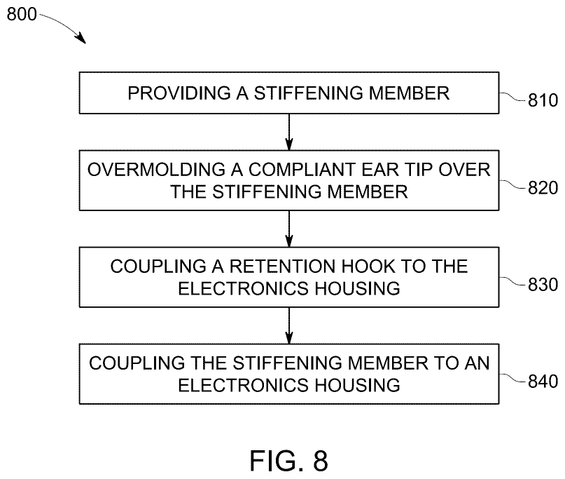

FIG. 8 is a flow chart of a method 800 for assembling an animal sound control device, according to some embodiments. At step 810, the method 800 includes providing a stiffening member. The stiffening member includes any and/or all features of the stiffening member(s) 316, 316a, 316b described above and in FIGS. 1-7B.

At step 820, the method 800 includes overmolding a compliant ear tip over the stiffening member. The compliant ear tip includes any and/or all features of the ear tip 300 described above and in FIGS. 1-7B. The overmolding includes positioning the stiffening member 316, 316a, 316b within a mold and injecting/extruding compliant material around the stiffening member 316, 316a, 316b to capture the stiffening member 316, 316a, 316b within the compliant ear tip 300. In some embodiments, the ear tip 300 is molded separate from the stiffening member 316, 316a, 316b and the stiffening member 316, 316a, 316b is positioned to be encapsulated by the compliant ear tip 300 (e.g., the compliant ear tip 300 is formed as two half-shells and the stiffening member 316, 316a, 316b is positioned therebetween).

At step 830, the method 800 includes coupling the stiffening member to an electronics housing. The electronics housing includes any and/or all features of the electronics housing 102 described above and in FIGS. 1-7B. In some embodiments, the sound tube 314 and the anchor port 312 are removably secured to the audio port 104 and the detent 106, respectively.

At step 840, the method 800 includes coupling a retention hook to the electronics housing. The retention hook includes any and/or all features of the retention hook 400 described above and in FIGS. 1-7B. In some embodiments, the retention ring 400 is removably secured to the channel 122 of the electronics housing 102.

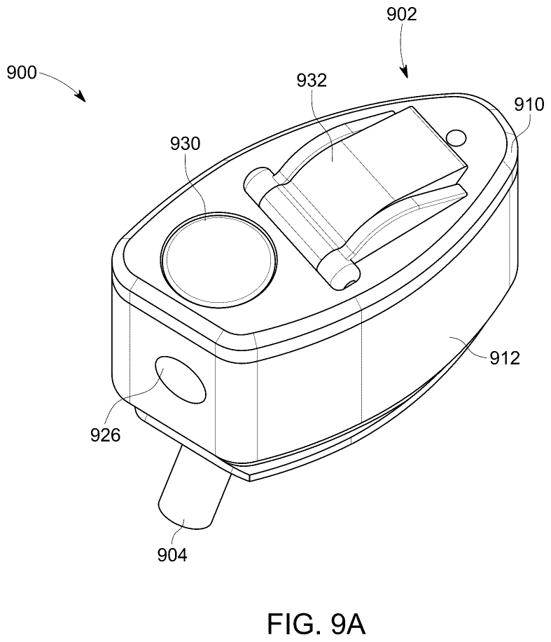

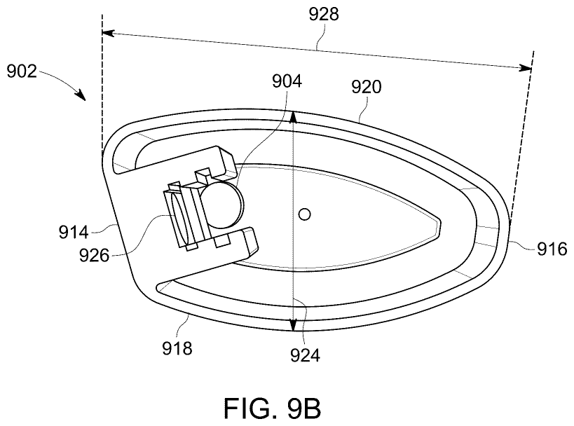

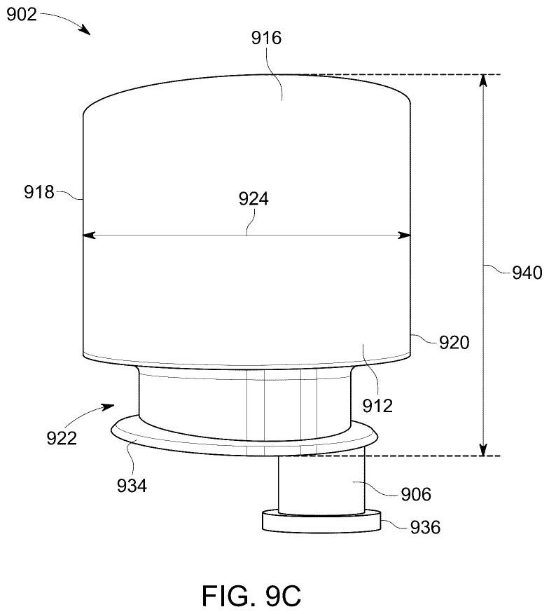

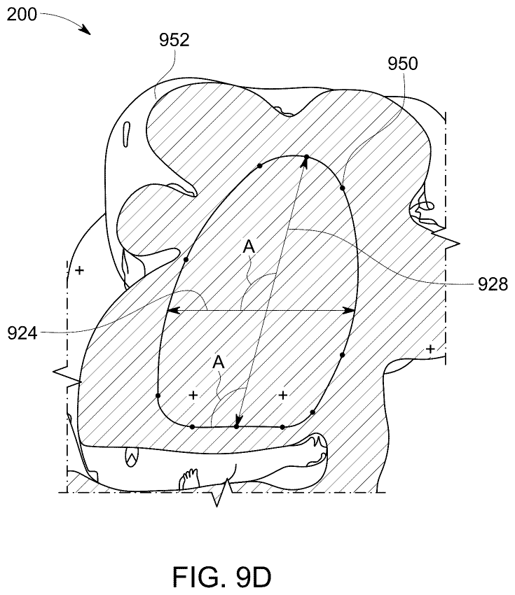

FIG. 9A is an isometric top view of an animal sound control device 900 including a magnet 930, according to some embodiments. In some embodiments, the animal sound control device 900 includes any and/or all features of the animal sound control device 100 and/or the animal sound control device 700 described above. The animal sound control device 900 includes a housing 902, an audio port 904, a cover 910, a body portion 912, and a hinged tab 932, according to some embodiments. FIG. 9B is a top view of the housing 902 of the animal sound control device 900 with the cover 910 removed, according to some embodiments. The housing 902 includes a first end 914, a second end 916, a first side wall 918, and a second side wall 920. The housing 902 extends a first length 928 between the first end 914 and the second end 916 and a second length 924 between the first side wall 918 and the second side wall 920. The housing 902 may include the audio port 904 and an audio jack port 926. FIG. 9C is a front side view of the second end 916 of the housing 902, according to some embodiments. The housing 902 includes the detent 906 including a ridge 936. The body portion 912 of the housing 902 extends a first height 940 and the second length 924 between the first side wall 918 and the second side wall 920. FIG. 9D is a diagrammatic top view of a cross-sectional profile 950 of the housing 902 within a canine ear canal 200 at a tragus-antihelix plane, according to some embodiments. The cross-sectional profile 950 of the housing 902 includes the first length 928 and the second length 924. The first length 928 (or the major axis) of the housing 902 is oriented at angle A relative to the second length 924 (or the minor axis) and the first end 914.

The housing 902 is configured to be received within a pinna of a canine ear canal. For instance, the body portion 912 of the housing 902, including the first end 914, the second end 916, the first side wall 918, and the second side wall 920, is shaped to be received within a pinna 952 of the canine ear canal 200. The housing 902 maximizes available volume within the pinna 952 to provide ample volume for electronics components and/or other features of the animal sound control device 900 (e.g., a battery, a microphone, a sound filter, etc.). Increasing volume within the housing 902 is beneficial, as it provides necessary volume for electronics components and systems within the animal sound control device 900 to increase functionality of the animal sound control device 900. Maximizing volume of the housing 902 within the pinna 952 may also improve retention of the animal sound control device 900 within the canine ear canal 200, as for instance, movement of the animal sound control device 900 is restricted.

The housing 902 may be configured to minimize direct contact with the canine ear canal 200. For instance, the body portion 912 of the housing 902, including the first end 914, the second end 916, the first side wall 918, and the second side wall 920, is shaped to conform to curvature of the pinna 952 without contacting the pinna 952. Direct contact between the housing 902 and the pinna 952 may cause discomfort and/or damage to the canine ear canal 200. The housing 902 is therefore configured to occupy a maximum available volume within the canine ear canal 200 while minimizing direct contact between the body portion 912 and the pinna 952.

The housing 902 occupies maximum available volume within the canine ear canal 200 while minimizing direct contact between the body portion 912 and the pinna 952 via a tapered profile (e.g., the first end 914 being wider than the second end 916) and/or via an angled profile (e.g., the angle A being complementary for the right and left ear canals). For instance, the front of the pinna 952 may be wider than a rear of the pinna 952. The first end 914 of the housing 902 is wider than the second end 916. In some embodiments, the angle A between the first length 928 (or the major axis) of the housing 902 and the second length 924 (or the minor axis) is within a range of 95° and 115°, and in some embodiments, the angle A is within a range of 100° and 110°. In some embodiments, the angle A is within a range of 100° and 105°. Due to the handed nature of canine ears, the right and left housing may be mirrored (e.g., a mirror image over a vertical plane) such that the respective first lengths 928 (or the major axes) of the right and left housings.

The body portion 912 of the housing 902 may include rounded edges to improve comfort. For instance, a sharp edge of the body portion 912 may cause damage and/or discomfort to the animal. In some embodiments, the minimum radius of curvature of any edge or surface of the body portion 912 (e.g., the transition between the first side wall 918 and the first end 914) is greater than 0.100 inches.

The first length 928 is greater than the second length 924, and in some embodiments, a ratio between the first length 928 and the second length 924 is between 4:3 and 2:1. The first length 928 is greater than the second length 924 because the pinna 952 is generally longer in one dimension (i.e., the major axis). Further, there are sensitive structures (e.g., ear canal structures with a high aspect ratio and/or low radius of curvature) adjacent to the minor axis that vary between canines, and therefore, the second length 924 is reduced (as compared to the first length 928) to avoid contact with these sensitive structures.

It should be noted that the canine ear canal 200 and the pinna 952 shown in FIG. 9D is an exemplary ear canal, and that each canine ear canal and/or canine pinna may include a unique geometry. For instance, the size of the canine (e.g., based on weight) may be a factor that influences canine ear canal size and geometry. The first length 928 and the second length 924 can be selected to fit within a bounding box for all canines of a certain weight range, e.g., a bounding box for canines between 41 pounds and 52 pounds can be created, and the cross-sectional profile 950 of the body portion 912 can fit within the bounding box.

The housing 902 defines a cavity within the body portion 912 to house one or more electronic components and/or other hearing components. The cavity may extend the first height 940 into the body portion 912. Keeping the electronics components as low as possible in the ear may be beneficial, as the electronics components can be secured within the structure of the canine ear canal 200 to reduce the possibility of dislodgement during use. The first height 940 is greater than the second length 924, according to some embodiments. The first length 928 is greater than the first height 940, according to some embodiments.

The housing 902 includes one or more retention features configured to anchor the housing 902 within the canine ear canal 200. For instance, the channel 922 and/or the ridge 934 may be configured to secure a retention ring (e.g., the retention hook 400 shown in FIGS. 4A-C). The retention ring can be received within the channel 922 and abut the ridge 934 to secure the housing 902 to the antihelix, tragus, and/or other ear canal features. The detent 906 and/or the ridge 936 may be configured to secure a stiffening member (e.g., the stiffening member(s) 316, 316a, 316b shown in FIGS. 3A-F and/or FIGS. 5A-B).

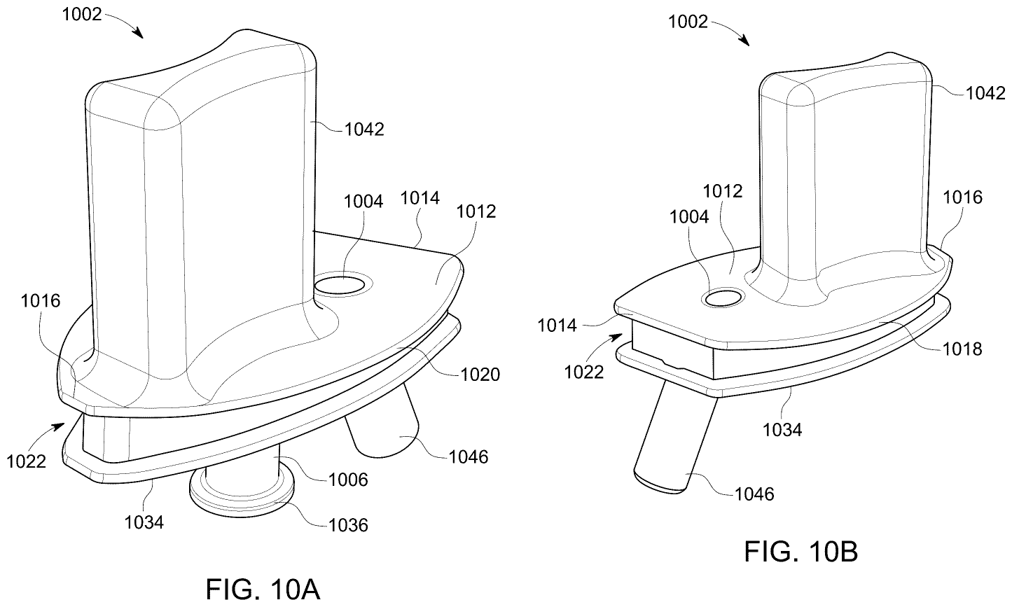

FIG. 10A is an isometric front view of an animal sound control device 1002, according to some embodiments. The animal sound control device 1002 may be a mechanical sound filter device, e.g., not including any electronic sound filtering features. FIG. 10B is an isometric rear view of the animal sound control device 1002, according to some embodiments. The animal sound control device 1002 includes a body portion 1012 including a first end 1014, a second end 1016, a first side wall 1018, and a second side wall 1020. The animal sound control device 1002 extends a first length between the first end 1014 and the second end 1016 and a second length between the first side wall 1018 and the second side wall 1020. The first length and the second length of the animal sound control device 1002 may be similar to and/or identical to the second length 924 and the first length 928. In some embodiments, the body portion 1012 may include any and/or all features (or dimensions) of the body portion 912 described above. The animal sound control device 1002 may include the audio port 1004. The animal sound control device 1002 includes a channel 1022, a ridge 1034, and a detent 1006 including a ridge 1036 extending from the bottom end of the animal sound control device 1002. A tab 1042 extends from an upper surface of the body portion 1012, according to some embodiments. The tab 1042 can be used to remove the animal sound control device 1002 from the canine ear canal 200, e.g., by providing an accessible engagement surface for the canine owner to remove the animal sound control device 1002. In some embodiments, a mechanical filter (e.g., foam or other sound dampening material) can be insert into the audio port 1004 and/or an audio tube 1046 to provide sound dampening.

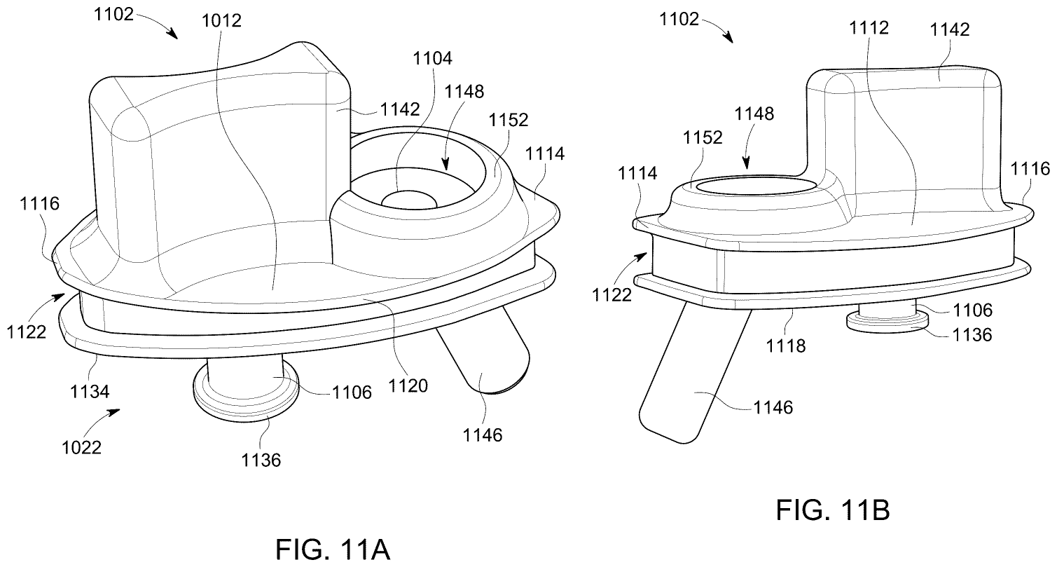

FIG. 11A is an isometric front view of an animal sound control device 1102, according to some embodiments. The animal sound control device 1102 may be a mechanical sound filter device, e.g., not including any electronic sound filtering features. FIG. 11B is an isometric rear view of the animal sound control device 1102, according to some embodiments. The animal sound control device 1102 includes a body portion 1112 including a first end 1114, a second end 1116, a first side wall 1118, and a second side wall 1120. The animal sound control device 1102 extends a first length between the first end 1114 and the second end 1116 and a second length between the first side wall 1118 and the second side wall 1120. The first length and the second length of the animal sound control device 1102 may be similar to and/or identical to the second length 924 and the first length 928. In some embodiments, the body portion 1112 may include any and/or all features (or dimensions) of the body portion 912 described above. The animal sound control device 1102 may include the audio port 1104 and an audio jack port 1126. The animal sound control device 1102 includes a channel 1122, a ridge 1134, and a detent 1106 including a ridge 1136 extending from the bottom end of the animal sound control device 1102. A tab 1142 extends from an upper surface of the body portion 1112, according to some embodiments. The tab 1142 can be used to remove the animal sound control device 1102 from the canine ear canal 200, e.g., by providing an accessible engagement surface for the canine owner to remove the animal sound control device 1102. In some embodiments, a mechanical filter (e.g., foam or other sound dampening material) can be insert into the audio port 1104 and/or an audio tube 1146 to provide sound dampening. A cavity 1148 disposed on the upper surface of the body portion 1112 and surround by a side wall 1152 may be configured to house a mechanical filter. The audio port 1104 may be positioned within the cavity 1148.

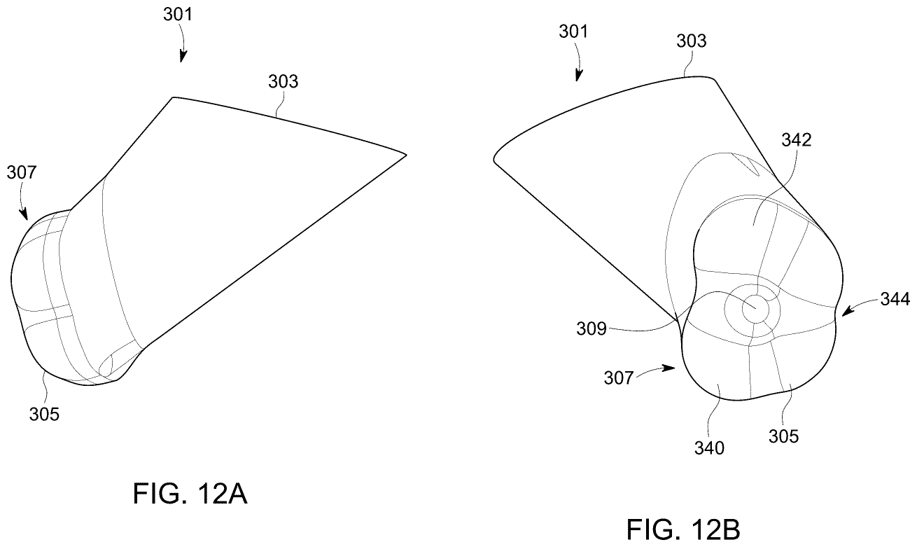

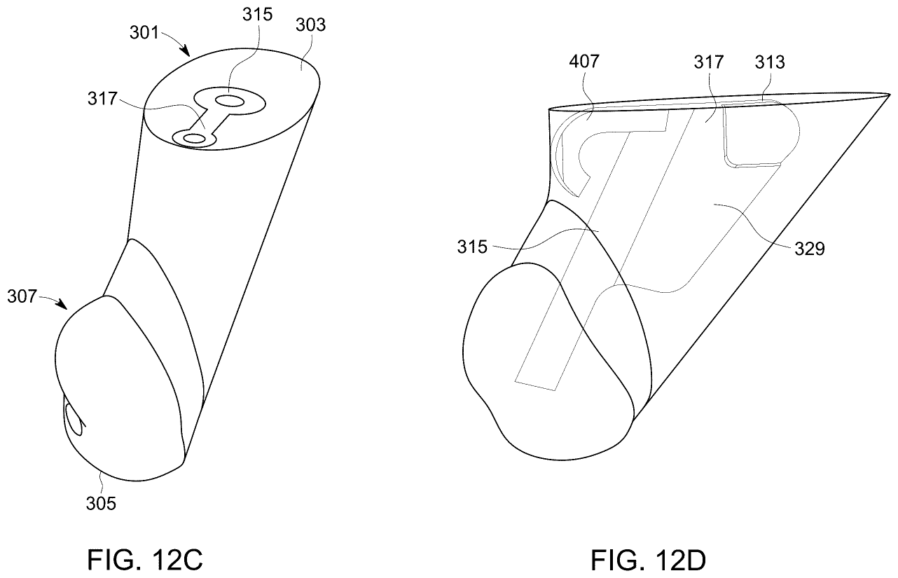

FIG. 12A is a side view of an ear tip 301 for an animal sound control device, according to some embodiments. The ear tip 301 includes a proximal end 303 and a distal end 305 with a sealing head 307 positioned at the distal end 305. FIG. 12B is a bottom view of the ear tip 301 an animal sound control device, according to some embodiments. The ear tip 301 may include an audio transmission aperture 309 on the scaling head 307. The sealing head 307 may include a first bulb 340 and a second bulb 342 with a depressed channel 344 extending therebetween. The audio transmission aperture 309 may be located between the first bulb 340 and the second bulb 342. FIG. 12C is an isometric side view of the ear tip 301 for an animal sound control device including a stiffening member 317 and a sound tube 315, according to some embodiments. FIG. 12D is a side view of the stiffening member 317 of the ear tip 301, according to some embodiments. The stiffening member 317 includes the sound tube 315, a stiffening rib 329, an anchor port 313, and a canal heel 407. The canal heel 407 may include a tragus shoe configured to engage a tragus of an animal ear (e.g., a tragus of a canine) to limit travel of the animal sound control device 100 in the animal ear. The canal heel 407 may optionally replace the canal heel 406 on the retention hook 400. It should be noted that the ear tip 301 of FIGS. 12A-D may include any and/or all features of the ear tip 300 described above in FIGS. 3A-F. For instance, the sealing head 307 may include any and/or all features of the scaling head 306, the stiffening member 317 may include any and/or all features of the stiffening member 316, and the sound tube 315 may include any and/or all features of the sound tube 314. The sealing head 307 may be configured to provide a seal at the second sealing plane 211 of the canine ear canal 200.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. An animal sound control device, comprising:
an ear tip formed of a resilient elastomeric material, the ear tip including:
an elongate body extending between a proximal end and a distal end,
a sealing head positioned on the elongate body, and
a stiffening member extending through the elongate body between the proximal end and the distal end.

2. The animal sound control device of claim 1, wherein the ear tip includes an audio transmission aperture extending within at least a portion of the elongate body, wherein the stiffening member includes a sound tube positioned within the audio transmission aperture, wherein the stiffening member is formed of a semi-rigid polymer material.

3. The animal sound control device of claim 1, wherein the sealing head is positioned at the distal end of the ear tip, and wherein a sealing head diameter is greater than an elongate body diameter.

4. The animal sound control device of claim 2, further comprising:
a housing secured to the proximal end of the ear tip.

5. The animal sound control device of claim 4, wherein the housing includes electronics components disposed therein, wherein the electronics components include a battery and a speaker for providing audio to the audio transmission aperture, wherein the electronic components within the housing include an acoustic filter to reduce a magnitude of audio.

6. The animal sound control device of claim 4, wherein the housing includes a housing including a body portion configured to be received within a canine ear canal, the body portion including:
a major axis extending between a first end and a second end,
a minor axis extending between a first side wall and a second side wall,
wherein the major axis includes a first length greater than a second length of the minor axis.

7. The animal sound control device of claim 4, further comprising a retention hook secured to the housing, wherein the retention hook is configured to engage one or more cartilage features of an animal ear to secure the animal sound control device in-ear of an animal.

8. The animal sound control device of claim 3, wherein the elongate body extends along a cylindrical axis between the proximal end and the distal end, wherein the sealing head diameter is positioned on a sealing head plane, wherein an angle between the cylindrical axis and the sealing head plane is non-orthogonal.

9. The animal sound control device of claim 8, wherein the stiffening member includes a distal head positioned parallel to the sealing head plane, wherein a distal end of the sound tube is positioned on the distal head.

10. An in-ear animal sound control device, comprising:
an ear tip formed of a compliant first material, the ear tip including:
an elongate body extending between a proximal end and a distal end,
a sealing head positioned on the elongate body; and
a retention hook formed of a compliant second material positioned near the proximal end of the ear tip, the retention hook configured to engage one or more cartilage features of an animal ear to secure the in-ear animal sound control device in-ear of an animal.

11. The in-ear animal sound control device of claim 10, wherein the retention hook includes an antihelix hook configured to engage an antihelix of the animal ear and a tragus shoe configured to be received within a tragus of the animal ear.

12. The in-ear animal sound control device of claim 10, wherein the retention hook includes a tab to facilitate grasping for removal of the in-ear animal sound control device.

13. The in-ear animal sound control device of claim 10, further comprising an electronics housing including one or more electronic components therein, wherein the electronics housing is detachably secured to a retention ring of the retention hook, wherein the electronics housing includes a ridge to receive the retention ring therein, wherein the electronics housing is removably coupled with the retention hook.

14. The in-ear animal sound control device of claim 10, wherein the ear tip includes a stiffening member extending through the elongate body between the proximal end and the distal end, the stiffening member including a sound tube, wherein the stiffening member is formed of a third material, the third material having a durometer greater than the compliant first material.

15. An animal sound control system, comprising:
a foam ear tip including:
an elongate body extending between a proximal end and a distal end,
a sealing head positioned on the elongate body, and
an audio transmission aperture extending within a least a portion of the elongate body;
an electronics housing positioned near the proximal end of the foam ear tip, the electronics housing including electronics components disposed therein.

16. The animal sound control system of claim 15, further comprising:
a retention hook including a compliant body securable to the electronics housing, the retention hook configured to engage one or more cartilage features of an animal ear to secure the animal sound control system in-ear of an animal.

17. The animal sound control system of claim 16, further comprising:
a stiffening rib disposed within the elongate body of the foam ear tip.

18. The animal sound control system of claim 17, wherein the stiffening rib extends from the proximal end of the foam ear tip to the sealing head to provide structural support for the foam ear tip in a direction parallel to a cylindrical axis of the elongate body.

19. The animal sound control system of claim 15, wherein the foam ear tip includes a concentric barb positioned between the proximal end and the sealing head to provide structural stability for the elongate body and provide a secondary sealing surface.

20. The animal sound control system of claim 15, wherein the sealing head forms a continuous sealing surface in a region adjacent to a bend between a vertical ear canal and a horizonal ear canal of an animal.
